(19) United States
(12) Patent Application Publication
(10) Pub. No.: US 2017/0007534 A1
(43) Pub. Date: Jan. 12, 2017

(54) CHAYOTE FRUIT CELL FLUID, PREPARATION METHOD THEREOF, SKINCARE AND WRINKLE-SMOOTHING COMPOSITION INCLUDING THE SAME, AND METHOD OF PREPARING WRINKLE-SMOOTHING COMPOSITION

(71) Applicant: Maxigen Biotech Inc., New Taipei City (TW)

(72) Inventors: Hui-Kang WANG, San Diego, CA (US); Yen-Jen SHIH, Taipei City (TW); Chih-Hao CHANG, Taipei City (TW)

(21) Appl. No.: 15/045,850

(22) Filed: Feb. 17, 2016

(30) Foreign Application Priority Data
Jul. 7, 2015 (TW) 104122079

(51) Int. Cl. A61K 8/97; A61Q 19/08; A61K 8/06; A61Q 19/00

(52) U.S. Cl. CPC A61K 8/97; A61Q 19/00; A61Q 19/08; A61K 8/068; A61K 2800/805

(57) ABSTRACT

A chayote fruit cell fluid, an extraction method of the chayote fruit cell fluid, a skincare and wrinkle-smoothing composition including the chayote fruit cell fluid and a method of preparing wrinkle-smoothing composition are disclosed. The extraction method of the chayote fruit cell fluid includes: squeezing a chayote fruit to obtain chayote fruit juice; filtering the chayote fruit juice for a first time; setting the filtered chayote fruit juice at room temperature for a period; and filtering the resultant chayote fruit juice for a second time to obtain the chayote fruit cell fluid. The method extracts the chayote fruit cell fluid from fresh chayote in a low-temperature process to provide better skincare and wrinkle-smoothing effects and maximize the natural activity of the chayote fruit.

### CROSS-REFERENCE TO RELATED APPLICATION

This application claims priority from Taiwan Patent Application No. 104122079, filed on Jul. 7, 2015, in Taiwan Intellectual Property Office, the contents of which are hereby incorporated by reference in their entirety for all purposes.

### BACKGROUND OF THE INVENTION

1. Field of the Invention

The present invention generally relates to a chayote fruit cell fluid, an extraction method of the chayote fruit cell fluid, a skincare and wrinkle-smoothing composition including the chayote fruit cell fluid, and a use of the chayote fruit cell fluid, and more particularly to a fruit cell fluid extracted from a chayote fruit by a novel extraction method, and a skincare and wrinkle-smoothing composition including the extracted chayote fruit cell fluid, and the use of the fruit cell fluid in skincare products, cosmetics, and pharmaceutical products.

2. Description of the Related Art

Since population ageing is a global trend, and older people have significant changes of skin appearance, therefore related products for delaying skin aging specially attract attention. Wrinkle is one of the characteristics of skin aging, and some of the common factors of causing wrinkles are described below:

1. Psychological Factor: Bad mood results in poor blood circulation of human body, and a sad, depressed, irritable, or solitary person always has a facial expression of anxiety, tension and stiffness, and the face muscles are affected to produce horizontal and vertical wrinkles and show signs of aging.

2. Bad Habits: People frequently having insufficient sleep, smoking heavily, and lacking exercises usually have poor skin metabolism and regulation which will result in haggard face, skin aging, and wrinkles.

3. Insufficient Water: Water is an important component of skin and also a necessary substance for skin metabolism. In general, if the water content of skin is less than 10%, the skin will become dry, rough and loose. Over a long period of time, the dry, rough and loose skin will lead to wrinkles.

4. Exposure to Sunlight: The skin after being exposed to sunlight for a long time will become dry and thin and lose elasticity. If elastic fibers and keratin fibers lose their normal functions, the skin will also become loosened and wrinkled gradually.

5. Improper Diet: Long-term unbalanced diet and poor nutrition will also lead to poor nutrition of skin and muscle tissues and rough and loose skin, and accelerate the production of wrinkles.

6. Improper Use of Cosmetics: Improper use of cosmetics will damage the texture of skin and reduce skin fibers, so that the water content and elasticity of skin drops, and wrinkles occur. A too-thick foundation also will cause fine wrinkles occurred on a face.

To prevent and improve wrinkles, maintaining skin water content over 10% is also an important step for skincare and wrinkle prevention in addition to sun protection and maintaining a healthy lifestyle. The water content of healthy skin should be maintained at approximately 15˜20%, and a too low water content of the skin may cause sensitive skin. The water content must be more than at least 30% to get a crystal-clear skin. Therefore, the step of maintaining the water content of the skin or moisturizing the skin plays an important role on resisting wrinkles and aging or reducing skin allergy.

In addition, the aging phenomenon caused by ultraviolet light is called photoaging, wherein the quantity of metalloproteinases of a dermis (matrix metalloproteinase, MMP) is increased by the phosphorylation of mitogen activated protein kinases. The metalloproteinase will decompose collagen to reduce the collagen content of skin. Without the support by collagen, the skin will become loosened and lack of elasticity, and cuticle will be proliferated excessively, and wrinkles are increased. At present, there are approximately 21 types of animal collagens found, and collagen Type I has the most quantity and broadest function. Different collagens are defined according to different tissues. In skin tissues, collagen Type I occupies approximately 80%, and collagen Type III occupies approximately 20%. If the activity of metalloproteinase particularly Type I can be inhabited and/or its formation can be reduced, then the effect of improving and enhancing the skin quality can be achieved.

Based on the consideration of safety and natural property, related technologies of developing anti-aging agents from natural products are attempted. However, the development of natural, safe, hypoallergenic skincare and wrinkle-smoothing products with better effect is still needed.

Chayote fruit (Sechium edule) is cultivated all over the world including China, and its fruit, leaf, bud, stem and root are generally used as food, and the fruit is nutritious and rich in protein, fat, cellulose, carbohydrates, vitamin C, riboflavin, calcium, phosphorous, iron, etc, so that the fruit can be used for cooking or eaten as a fruit. Researches on chayote fruits show that the chayote fruit has the antibacterial, antioxidant, anti-diabetic, anti-ulcer, anti-tumor, epilepsy, liver and kidney protections, anti-hypertensive, anti-mutation, inhibition of trypsin and other effects.

Literatures pointed out that a skin lotion containing a chayote fruit extract is capable of moisturizing skin up to 20% and providing an effect of removing oxygen free radicals. Literatures also indicated that dried fruits extracted by heating with water or ethanol are used as research materials. The present invention provides a novel extraction method of a chayote fruit cell fluid, and this method extracts a chayote fruit cell fluid with a better wrinkle-smoothing effect from fresh chayote fruits by low-temperature and room temperature processes to maximize the preservation of the natural activity of the chayote fruit.

### SUMMARY OF THE INVENTION

Therefore, it is a primary objective of the present invention to provide an extraction method of chayote fruit cell fluid, fresh chayote fruit cell fluid with a better wrinkle-smoothing effect resulting from the extraction method, a skincare and wrinkle-smoothing composition include the chayote fruit cell fluid, and a preparation method of the chayote fruit cell fluid for preparing the skincare and wrinkle-smoothing composition.

To achieve the foregoing objective, the present invention provides an extraction method of a chayote fruit cell fluid comprising the steps of: squeezing a chayote fruit to obtain a fresh chayote fruit juice; filtering the chayote fruit juice for a first time to remove fine and small fruit marcs; letting the filtered chayote fruit juice stand at room temperature for a period of time; and filtering the chayote fruit juice for a second time to obtain the chayote fruit cell fluid.

Preferably, the first filter is performed by using a 64-mesh nylon filter cloth, and the second filter is performed by using a filter paper or a centrifugal filter method.

Preferably, the period of standing is approximately 24 hours or more.

To achieve the other objective of the present invention, the present invention provides a chayote fruit cell fluid prepared by the aforementioned extraction method of the chayote fruit cell fluid.

Preferably, the chayote fruit cell fluid is analyzed by a high pressure liquid chromatography (HPLC) with the following conditions: Instrument: Agilent 1200 HPLC System; Injection Volume: 50 microliters; Analysis Column: Phenomenex Luna C18(2), 250×4.5 millimeters, 5 microns; Analysis Time: 45 minutes; Column Temperature: 30° C.; Mobile Phase: Solvent A: water and 10 mM of NH₄OAc, Solvent B: Acetonitrile and 0.1% of formic acid; Gradient: A: dropping from 100.0% to 0.0% in 45 minutes, B: rising from 0.0% to 100.0% in 45 minutes; Velocity of Flow: 1.0 ml/min; Measured Wavelength: 254 nm, and the obtained HPLC spectrum within the interval of 2˜15 minutes has absorption peaks with a retention time approximately equal to 2.506 minutes, 2.754 minutes, 2.986 minutes, 4.048 minutes, 6.438 minutes, and 8.358 minutes.

To achieve a further objective, the present invention provides a skincare and wrinkle-smoothing composition comprising 20-98.5% by weight of the chayote fruit cell fluid having a HPLC spectrum with the aforementioned characteristic, and a skin, cosmetically or pharmaceutically acceptable carrier.

Preferably, the carrier is a diluent and an excipient of a mask, a cream, an essence, a lotion, etc, and the carrier includes solutions, emulsions, gels, solids or liposomes.

Preferably, the emulsions include a microemulsion or a nanoemulsion.

To achieve a further objective, the present invention provides a method of skincare and wrinkle-smoothing, including applying the chayote fruit cell fluid or the skincare and wrinkle-smoothing composition mentioned above to skin.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The detailed structure, operating principle and effects of the present invention will now be described in more details hereinafter with reference to the accompanying drawings that show various embodiments of the invention as follows.

FIG. 1 is a flow chart of an extraction method of a chayote fruit cell fluid in accordance with an embodiment of the present invention;

FIG. 2 is a high pressure liquid chromatography (HPLC) spectrum of a chayote fruit cell fluid in accordance with an embodiment of the present invention;

FIG. 3 is a chart comparing a chayote fruit cell fluid of an embodiment of the present invention that suppresses the activity of matrix metalloproteinase-1 (MMP-1);

FIG. 4 shows photos of the skin condition of a volunteer (male, aged 62) before and after applying a composition 1 of an embodiment of the present invention;

FIG. 5 shows a photo and an ultrasonic skin scan of the skin condition of a volunteer (male, aged 62) before and after applying a composition 1 of an embodiment of the present invention;

FIG. 6 shows photos of the skin condition of a volunteer (female, aged 33) before and after applying a composition 2 (chayote fruit mask) of an embodiment of the present invention; and

FIG. 7 shows a photo and an ultrasonic skin scan of the skin condition of a volunteer (female, aged 33) before and after applying a composition 2 (chayote fruit mask) of an embodiment of the present invention.

### DETAILED DESCRIPTION OF THE PREFERRED EMBODIMENTS

The technical content of the present invention will become apparent by the detailed description of the following embodiments and the illustration of related drawings as follows. These embodiments and drawings are provided for the purpose of illustrating the present invention, but not intended for limiting the scope of the invention. For simplicity, same numerals are used for presenting respective same elements as described in the embodiments and illustrated in the drawings.

Unless otherwise defined, all terms used herein have the same meaning as commonly understood by one of ordinary skill in the art to which the present teachings belong. A singular form may include plural forms, unless there is a particular description contrary thereto.

It is noteworthy that the term “percentage by weight (wt. %)” refers to the percentage by weight of the component part in composition which added with a carrier and other constituents before applying the composition of the chayote fruit cell fluid.

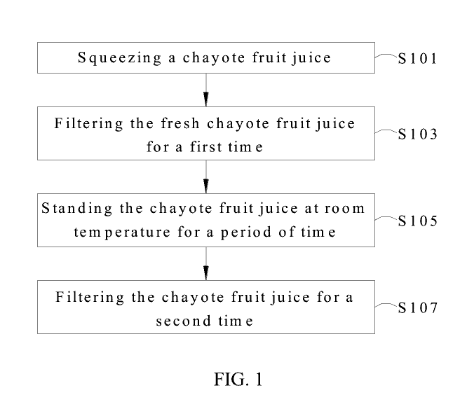

FIG. 1 is a flow chart of an method of extracting a chayote fruit cell fluid in accordance with an embodiment of the present invention

With reference to FIG. 1, the extraction method comprises the steps of: squeezing a chayote fruit juice (Step 101); filtering the fresh chayote fruit juice for a first time (Step 103); standing the chayote fruit juice at room temperature for a period of time (Step 105); and filtering the chayote fruit juice for a second time (Step 107) to obtain a chayote fruit cell fluid.

In Step 101, the fresh chayote fruit is cleaned by water and cut into pieces, preferably into approximately 2 cm strips, and put into a household or industrial fruit juice machine to squeeze a chayote fruit juice. In a preferred embodiment, the produced fruit marc may be squeezed for 2˜3 times to improve the yield.

In Step 103, the chayote fruit juice obtained in Step 101 is filtered for a first time by using a 64-mesh nylon filter to remove the small residues of the chayote fruit juice.

In Step 105, the chayote fruit juice in which the small residues are removed in Step 103 is stand at room temperature for over 24 hours, until the chayote fruit juice has precipitates.

In Step 107, a filter paper, a fine sand core funnel or a centrifugal method is used for a second filter to remove substances insoluble in water such as starch, chlorophyll, and cell wall settled in Step 105, so as to obtain a clear chayote fruit cell fluid. In a preferred embodiment, the second filter is performed by using a filter paper or a centrifugal method. In a preferred embodiment, the filter paper is a No. 2 filter paper, and the filtration is performed by air suction or gravitational filtration. In a preferred embodiment, the second filter is performed by centrifuge at 10,000 r.p.m. for 10 minutes.

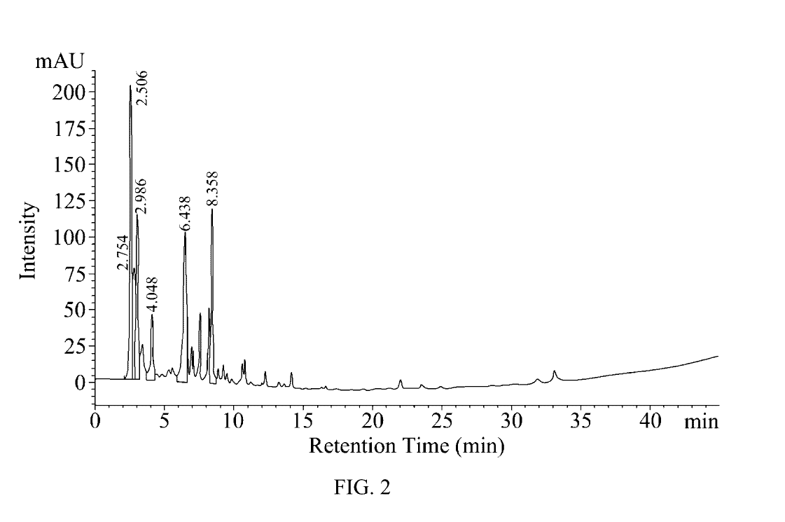

A preferred embodiment of the present invention further provides a chayote fruit cell fluid prepared by the aforementioned extraction method of chayote fruit cell fluid, wherein the chayote fruit cell fluid is analyzed by a high pressure liquid chromatography (HPLC) carried out in the following conditions: Instrument: Agilent 1200 HPLC System; Injection Volume: 50 microliters; Analysis Column: Phenomenex Luna C18(2), 250×4.5 millimeters, 5 microns; Analysis Time: 45 minutes; Column Temperature: 30° C.; Mobile Phase: Solvent A: water and 10 mM of NH₄OAc, Solvent B: Acetonitrile and 0.1% of formic acid; Gradient: A: dropping from 100.0% to 0.0% in 45 minutes, B: rising from 0.0% to 100.0% in 45 minutes; Velocity of Flow: 1.0 ml/min; Measured Wavelength: 254 nm, and the obtained HPLC spectrum within the interval of 2˜15 minutes has absorption peaks with a retention time approximately equal to 2.506 minutes, 2.754 minutes, 2.986 minutes, 4.048 minutes, 6.438 minutes, and 8.358 minutes as shown in FIG. 2.

A fluorescent substrate of collagenase (such as fluorogenic peptide substrate I) is used to evaluate the inhibition effect of the collagenase to the chayote fruit cell fluid of the present invention, wherein the collagenase is an extended-spectrum collagenase obtained by genetic reconstruction.

Firstly, 480 microliters of water is filled into a microcentrifuge tube, and 80 microliters of 10 times dilute buffer solution (prepared by 5 mL of 1 M trometamol (Tris) (pH7.8), 1 mL of 1M calcium chloride, 3.75 mL of 4M sodium chloride, and 0.25 mL of water) are added. Then, 80 microliters of chayote fruit cell fluid (SE-fresh), 80 microliters of collagenase (0.01 milligram/milliliter), and 80 microliters of fluorescent substrate (fluorogenic peptide substrate I, 10 μM)) are added thereto. After the obtained solution is mixed uniformly, the solution is put into an incubator at 37° C. and reacted for 20 hours. Then, a BioTek Synergy HTX multifunctional chemical analyzer (Excitation Light: 490 nm; Radiation Light: 520 nm) is used to test the absorption value of the solution. Finally, the inhibition rate of the chayote fruit cell fluid to the collagenase is calculated by the following formula, and the same procedure is used for carrying out the text for three times, and the average and standard deviation of the obtained inhabitation rates are calculated.

$$Inhabitation rate ( \% ) = \frac{( A - B ) - ( C - D )}{( A - B )} \times 100 \%$$

A: This solution contains collagenase but not the testing sample.
B: This solution does not contain collagenase and the testing sample.
C: This solution contains collagenase and the testing sample.
D: This solution does not contain collagenase and contains the testing sample.

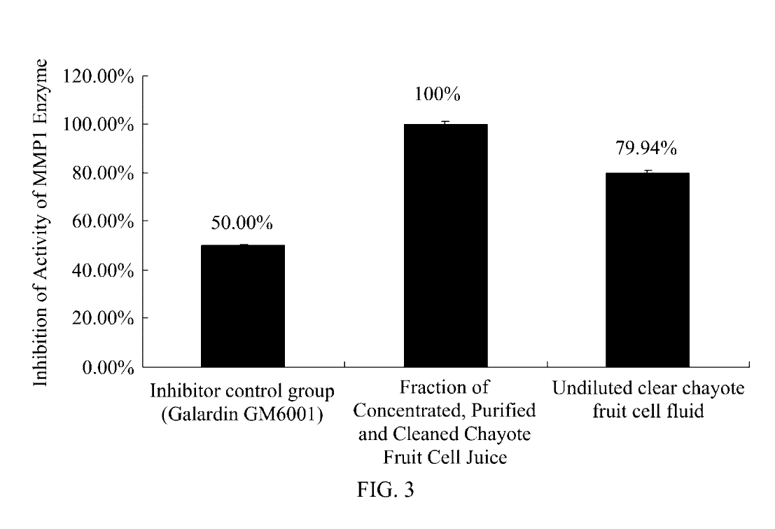

The experiment result as shown in FIG. 3 indicates that the chayote fruit cell fluid has a more significant inhabitation activity to MMP-1 than the control, and this experiment result shows that the chayote fruit cell fluid in accordance with this preferred embodiment of the present invention has a more significant effect on the inhibition of the activity of the matrix metalloproteinase-1 (MMP-1).

A preparation method of a skincare and wrinkle-smoothing composition is provided in a preferred embodiment of the present invention, wherein the chayote fruit cell fluid is used to prepare a composition with skincare and wrinkle-smoothing functions. In this preferred embodiment, the chayote fruit cell fluid having the HPLC spectrum as shown in FIG. 2 can be concentrated at room temperature by reducing pressure, frozen and dried, or prepared with any other conventional method to produce a composition with skincare and wrinkle-smoothing functions.

The composition in accordance with a preferred embodiment of the present invention comprises the aforementioned chayote fruit cell fluid and a skin, cosmetically or pharmaceutically acceptable carrier. The carrier of the present invention has no specific limitation, as long as it is not poisonous or is an inert carrier that does not react with the chayote fruit cell fluid of the present invention. The carrier is a diluent or excipient often used in a mask, a cream, an essence and a lotion. Such carrier includes, but not limited to, solutions, emulsions (such as a microemulsion and a nanoemulsion), gels, solids or liposomes. For example, the carrier includes water (such as deionzied water), a vegetable oil, a mineral oil, fats, alcohols, hydrocarbon oil, silicon oil, wax, or any combination or mixture of the above.

The carrier includes, but not limited to, solutions, emulsions (such as microemulsions and nanoemulsions), gels, solids or liposomes. The non-restrictive embodiment of the emulsion includes a water-in-oil emulsion, an oil-in-water emulsion, a polysiloxane-in-water emulsion, a water-in-polysiloxane emulsion, a wax-in-water emulsion, a three-phased water-oil-water emulsion or a cream, a gel or a microemulsion like substance. The emulsion may include an emulsifier such as a nonionic, anionic, or amphoteric surfactant.

In a preferred embodiment, the composition of the present invention comprises an additive applied to conventional skincare products, cosmetics, and pharmaceutical products including, but not limited to: a filter, an emulsifier, an antioxidant, a surfactant, a film forming agent, a chelator, a gallant, a thickener, a softener, a moisturizer, a vitamin, a mineral, a viscosity and/or rheologic modifier, a sunscreen agent, a keratolytic agent, a depigmenting agent, a retinoid, a hormone compound, an α-hydroxy acid, an α-keto acid, an antifungal agent, an antimicrobial agent, an antiviral agent, an analgesic agent, a lipid compound, an anti-allergic agent, a H1 or H2 antihistamine, an anti-inflammatory agent, an anti-irritant agent, an antiproliferative agent, an immune system booster, an immune system inhibitor, an anti-acyn agent, an anesthetic, a disinfectant, an insect repellant, a skin cooling compound, a skin protectant, a skin penetration enhancer, an exfoliating agent, a lubricant, an aromatic agent, a coloring agent, a depigmenting agent, a pigment fading agent, a preservative, a stabilizer, a pharmaceutical agent, a light stabilizer, a neutralizer, a collagenase inhibitor, an elastic protease inhibitor, a skin plump agent, a stripping agent, and any mixture of the above.

In another preferred embodiment, the composition of the present invention further comprises a plant extract or an animal extract, wherein the animal extract includes, but not limited to, collagen, water soluble collagen, water soluble protein, elastin, placenta, etc. The plant extract includes, but not limited to, aloe vera, chamomile, ginkgo, witch hazel, licorice root, sage, seaweed, Indian fir, green tea, rice, angelica root, angelica fungus, Avocado, oleander, etc.

In a preferred embodiment, the composition of the present invention comprises 98.5 wt. % or more of the aforementioned chayote fruit cell fluid and 1 wt. % of the carrier. Preferably, the composition of present invention comprises 20 wt. % or more of the aforementioned chayote fruit cell fluid and 80 wt. % of the carrier.

Wherein, the composition of the present invention is in form of detergent, cream, slurry, spray, aerosol, pie, ointment, essence, gel, paste, patch, pen-like object, wet tissue, mask, rod, foam, elixir, concentrated solution, etc. More specifically, the composition may be in form of emulsion or cream which is topical applied to a portion of skin having wrinkles and/or fine lines. Preferably, the composition may be applied directly to fine lines and/or wrinkles. The composition is applicable to fine lines and/or wrinkles of any skin surface including but not limited to the skin of face, neck, and/or hand.

The following non-restrictive embodiments are provided for persons having ordinary skill in the art to implement the present invention. It is intended that these embodiments are to be considered illustrative rather than restrictive, and numerous modifications and variations could be made thereto by those skilled in the art without departing from the scope and spirit of the invention set forth in the claims.

### Preferred Embodiment 1: Preparation of Composition 1

Step 1: Extract a chayote fruit cell fluid.

Rinse and clean 2,508 grams of fresh chayote fruit by water. Cut the chayote fruit into strips with a width of approximately 2 cm. Put the strips of chayote fruit into a Hurom HR808 slow grinder to squeeze a chayote fruit juice. Squeeze the produced fruit marc again for two times to improve the yield. Filter the chayote fruit juice by a 64-mesh nylon filter cloth to remove residues, so as to obtain 2,277 grams of chayote fruit juice. Let the chayote fruit juice stand at room temperature for 24 hours until substances including starch, chlorophyll, and cell wall of the chayote fruit juice which are insoluble in water are fully precipitated. Use No. 2 filter paper to filter the precipitate, so as to obtain 2,240 grams of the clear chayote fruit cell fluid.

Step 2: HPLC Analysis

(2) Testing Sample: Undiluted clear chayote fruit cell fluid

(3) HPLC Conditions

Instrument: Agilent 1200 HPLC system; Injection Volume: 50 microliters; Analysis Column: Phenomenex Luna C18(2), 250×4.5 millimeters, 5 microns; Analysis Time: 45 minutes; Column Temperature: 30° C.; Mobile Phase: solvent A: water and 10 mM of NH₄OAc, solvent B: acetonitrile and 0.1% formic acid, gradient: A (dropping from 100.0% to 0.0% in 45 minutes), B (rising from 0.0% to 100.0% in 45 minutes); Velocity of Flow: 1.0 ml/min; and Measured Wavelength: 254 nm.

With reference to FIG. 2 for the HPLC analysis results, the absorption peaks occur at the retention time of approximately 2.506 minutes, approximately 2.754 minutes, approximately 2.986 minutes, approximately 4.048 minutes, approximately 6.438 minutes, approximately 8.358 minutes.

Step 3. Preparation of Composition 1

Add 0.75 grams of carbomer to 98.5 grams of the chayote fruit cell fluid, and mixture of the carbomer and the cell fluid added 0.75 grams of triethanolamine thereto is fully expanded and moisturized, and stir the mixture uniformly to prepare the composition 1.

Observe the Effect of Improving the Skin Quality after a Volunteer has Applied the Composition 1 to Skin

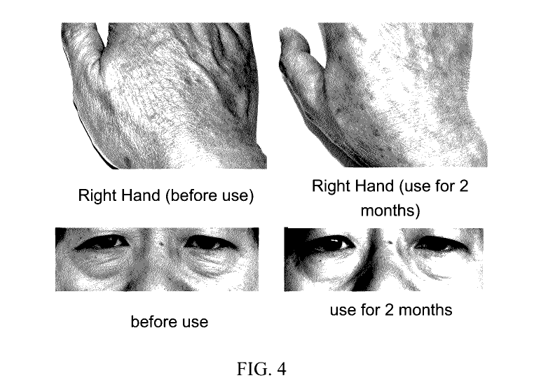

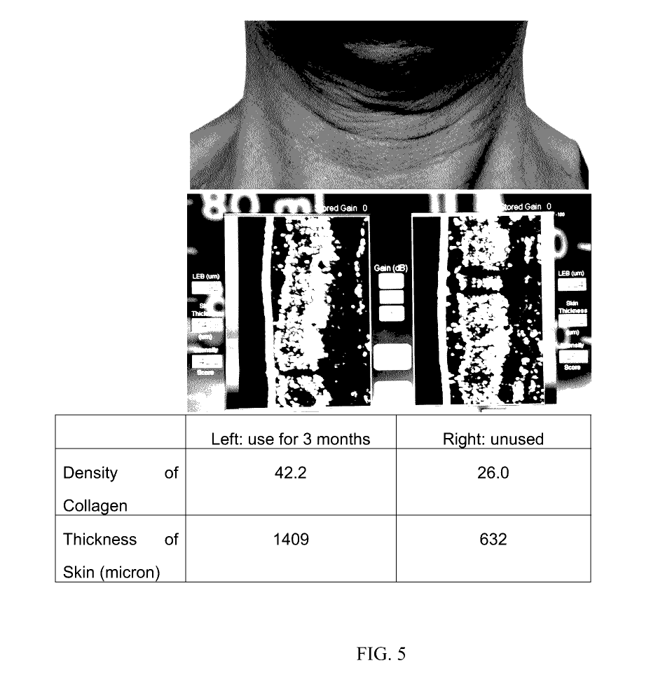

With reference to FIGS. 4 and 5, the skin of the volunteer's hands, face and neck are significantly improved, the skin elasticity is recovered, and the overall skin appearance is improved after using the composition 1. In FIG. 4, it is apparent that, before the use of the composition 1, the volunteer's right hand and face are redder and the veins are bluer, In FIG. 5, it is apparent that, after using for 3 months, the light spots (light green color) are markedly increased. It proves that the composition 1 increases the density of subcutaneous collagen and the thickness of skin significantly.

### Preferred Embodiment 2: Preparation of Composition 2

Add 76.75 grams of water to 20 grams of chayote fruit cell fluid for dilution. Add 1 gram of pentylene glycol, 0.25 gram of xanthan gum, and 2 grams of Leuconostoc/Radish Root Ferment Filtrate, and mix them uniformly to produce a chayote fruit facial mask liquid. Fold a mask and put it in an aluminum bag. Pour 25 grams of the chayote fruit facial mask liquid into the aluminum bag to complete the manufacture of the chayote fruit mask (composition 2).

Observe the Effect of Improving the Skin Quality after a Volunteer has Applied the Chayote Fruit Mask (Composition 2) to Skin.

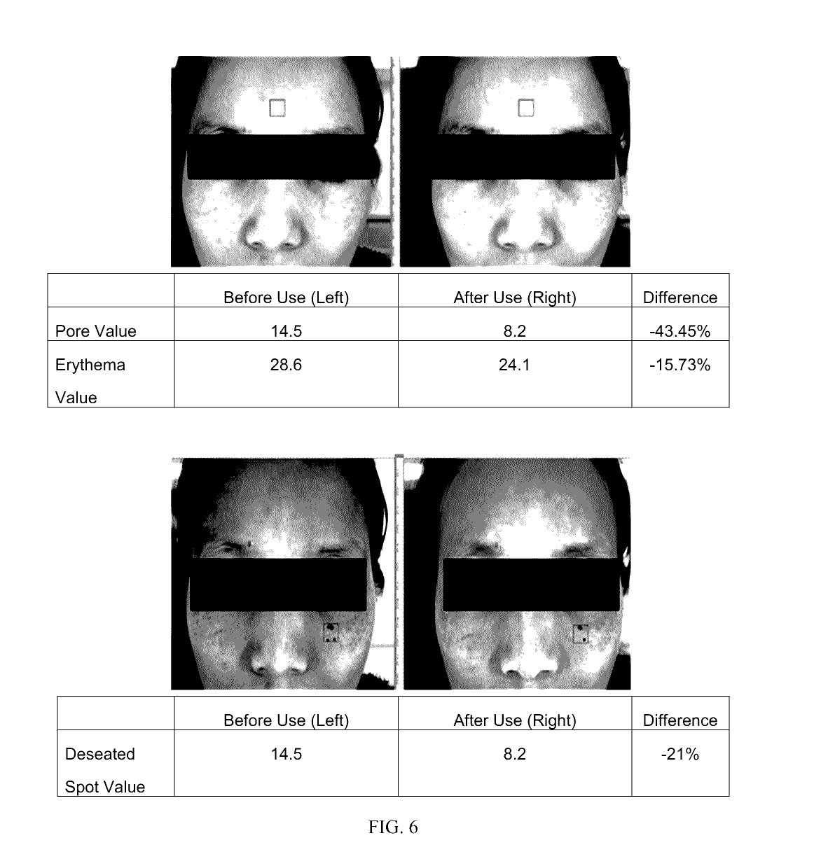

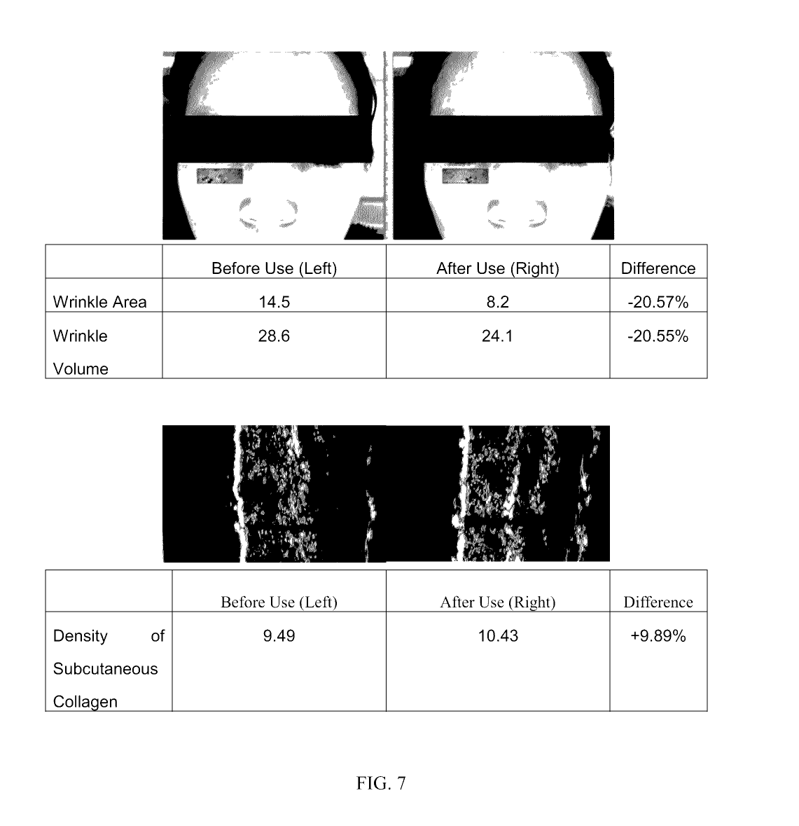

After a volunteer (female, aged 33) has completed washing her face and applied the chayote fruit mask for 15 minutes, the volunteer tear away the mask and wait for 5 minutes until the facial mask liquid is absorbed completely. With reference to FIGS. 6 and 7 for the testing results of the skin tested by a full face skin analyzer (IRV Derma) and an ultrasonic skin scanner (Cortex Dermascan USB) before and after the use of composition 2 respectively, the skin pores, erythema, and deseated spot are reduced significantly, and the wrinkles are smoothed, after the volunteer has applied the composition 2. The pore value, the erythema value and the deseated spot value are analysis results of the skin in the red box. In FIG. 6, it is apparent that, before the use of the composition 2, the volunteer's face is redder and the deseated spot which indicated by the dark red spots are more. In FIG. 7, it is apparent that, after using the composition 2, the light spots (light green color) are markedly increased, and the wrinkle which represented by light pink is decrease. It proves that the density of subcutaneous collagen is increased.

Therefore, the composition of the chayote fruit cell fluid obtained by the extraction method of the present invention has a notable wrinkle-smoothing effect.

While the means of specific embodiments in present invention has been described by reference drawings, numerous modifications and variations could be made thereto by those skilled in the art without departing from the scope and spirit of the invention set forth in the claims. The modifications and variations should in a range limited by the specification of the present invention.

1. A method of extracting a chayote fruit cell fluid, comprising the steps of squeezing a chayote fruit to obtain a chayote fruit juice;
filtering the chayote fruit juice for a first time;
standing the chayote fruit juice at room temperature for a period of time; and
filtering the chayote fruit juice for a second time to obtain the chayote fruit cell fluid.

2. The method according to claim 1, wherein the first filter is performed by using a 64-mesh filter.

3. The method according to claim 1, wherein the second filter is performed by using a filter paper or a centrifugal method.

4. The method according to claim 1, wherein the period of standing is approximately 24 hours or more.

5. A chayote fruit cell fluid, manufactured by the method according to claim 1.

6. The chayote fruit cell fluid according to claim 5, wherein the chayote fruit cell fluid is analyzed by a high pressure liquid chromatography (HPLC) with the following conditions: instrument: Agilent 1200 HPLC System; Injection Volume: 50 microliters; Analysis Column: Phenomenex Luna C 18(2), 250×4.5 millimeters, 5 microns; Analysis Time: 45 minutes; Column Temperature: 30° C.; Mobile Phase: Solvent A: water and 10 mM of NH₄OAc, Solvent B: Acetonitrile and 0.1% of formic acid; Gradient: A: dropping from 100.0% to 0.0% in 45 minutes, B: rising from 0.0% to 100.0% in 45 minutes; Velocity of Flow: 1.0 ml/min; Measured Wavelength: 254 nm, and the obtained HPLC spectrum within the interval of 2˜15 minutes has absorption peaks with a retention time approximately equal to 2.506 minutes, 2.754 minutes, 2.986 minutes, 4.048 minutes, 6.438 minutes, and 8.358 minutes.

7. A skincare and wrinkle-smoothing composition, comprising 20% to 98.5% by weight of the chayote fruit cell fluid according to claim 6, and a skin, cosmetically or pharmaceutically acceptable carrier.

8. The skincare and wrinkle-smoothing composition according to claim 7, wherein the carrier includes solutions, emulsions, gels, solids or liposomes.

9. The skincare and wrinkle-smoothing composition according to claim 8, wherein the emulsions includes a microemulsion or a nanoemulsion.

10. A method of skincare and wrinkle-smoothing, including applying the chayote fruit cell fluid according to claim 5 to skin.

11. A method of skincare and wrinkle-smoothing, including applying the skincare and wrinkle-smoothing composition according to claim 7 to skin.
